(19) United States
(12) Patent Application Publication
(10) Pub. No.: US 2014/0335180 A1
(43) Pub. Date: Nov. 13, 2014

(54) PRODUCTS FOR ORAL ADMINISTRATION COMPRISING EXTRACTS OF PUNICA GRANATUM (POMEGRANATE), INTENDED FOR A PET, AND APPLICATIONS OF SAME

(71) Applicant: VIRBAC SA, Carros (FR)

(72) Inventors: Patricia Monginoux, Villeneuve Loubet (FR); Hugues Gatto, Saint Paul (FR); Christian Karst, Biot (FR); Ferdinand Rudolf Waldenberger, Vienna (AU)

(73) Assignee: VIRBAC SA, Carros (FR)

(21) Appl. No.: 14/357,837

(22) PCT Filed: Nov. 8, 2012
(86) PCT No.: PCT/FR2012/000447

§ 371 Date: May 13, 2014

(30) Foreign Application Priority Data
Nov. 14, 2011 (FR) 11/03450

(51) Int. Cl. A61K 36/185

(52) U.S. Cl. CPC A61K 36/185
USPC 424/479; 424/725

(57) ABSTRACT

The invention relates to a product for oral administration intended for a pet, comprising extracts of punica granatum (pomegranate). The invention is characterised in that the invention further comprises at least two additional antioxidant compounds chosen from:— soya isoflavone extracts;— L-carnitine; and— taurine.

### FIELD OF THE INVENTION

The invention relates to a product for oral administration intended for a pet, comprising extracts of punica granatum (pomegranate) associated with further antioxidants, in the field of complete foods, supplementary (dietary supplements) or health foods. More particularly, the invention relates to a product for oral administration intended for a pet, comprising extracts of punica granatum (pomegranate) associated with further antioxidants for protecting, treating or helping control cardiovascular disease.

### BACKGROUND OF THE INVENTION

The increase in the life expectancy of pets such as for example, dogs and cats, is now commonplace due to many advances in recent decades, particularly in the fields of animal nutrition, preventive healthcare, medicine or surgery.

In dogs for example, heart disease is considered to be the fourth most frequent cause of mortality. Heart conditions can be classified according to whether they are innate, hereditary (congenital diseases) or acquired. The acquired diseases are essentially valve failure and dilated cardiomyopathy. The latter may affect any dog breed. Nevertheless, some breeds are more predisposed than others. Furthermore, these diseases also have a high incidence in older dogs.

Valve failure represents up to 75% of cases of heart disease in dogs. It is thus the disease most frequently encountered in this animal.

Valve failure is generally caused by progressive myxomatous degeneration of the atrioventricular valves which is a factor in the pathogenesis of the disease and increased cellular oxygen demand. The risk of mitral dysfunction increases with age. It has been demonstrated that one third of dogs of predisposed breeds are small in size and have a heart murmur characteristic of valve failure from the age of 7 years. Three-quarters of cases of heart failure observed in dogs are secondary to this primary valve disease.

Dilated cardiomyopathy (DCM) is the second most frequent acquired disease, after valve failure in dogs. It tends to affect large dogs and may also be the result of the progression of valve failure.

In cats, such heart conditions are also found. The most common heart condition is hypertrophic cardiomyopathy. This condition mainly affects males and may occur at any stage of life, with a major incidence at around the age of 5-7 years. Further conditions such as, for example, dilated cardiomyopathy and hyperthyroid heart failure also affect cats.

Experimental studies (Cesselli, D. et al. Oxidative stress-mediated cardiac cell death is a major determinant of ventricular dysfunction and failure in dog dilated cardiomyopathy. Circ Res 89, 279-86 (2001)) have demonstrated an important role of oxidative stress in the development of canine cardiovascular disease. More specifically, it has been demonstrated that oxidative stress induced damage in a plurality of types of cardiovascular cells, capable of inducing the initiation and progression of heart disease.

Recent data thus suggest that oxidative stress is involved in the regulation of cardiomyocyte apoptosis (programmed cell death). These deregulations are capable of modifying the antioxidant reactivity of the cells and, consequently, increase heart failure severity.

Indeed, accelerated vascular endothelial cell apoptosis impedes vascularization and adds to the development and progression of vascular disease. Recent data suggest that the increase in the endothelial cell renewal rate occurs in response to increased apoptosis rates. These two processes appear to impede the integrity of the endothelial cell monolayer and cause functional alterations including an increase in vascular permeability and a change of secretion profile promoting vasoconstrictive, pro-adhesive, pro-thrombotic and pro-inflammatory activities resulting in fibrosis and progressive loss of valve elasticity and tightness.

Given the prevalence of heart conditions in animals (particularly valve failure), and considering that oxidative stress plays a major role in the development and progression of heart disease, the possibility of regulating this oxidative stress with exogenous compounds has thus been considered in more detail. The advantages of reducing oxidative stress in conjunction with physiopathological anomalies have been demonstrated in humans, and considerable work has been done to identify and characterize the substance liable to protect endothelial cells from oxidative stress.

However, the effects of antioxidants differ according to the species. Indeed, as reported by Ram, J. I. & Hiebert, L. M. Vitamin E protects porcine but not bovine cultured aortic endothelial cells from oxygen-derived free radical injury due to hydrogen peroxide. Cell Biol Toxicol 20, 55-67 (2004), it would appear that the protective effects provided by antioxidant substances are species-dependent. It is thus not possible to extrapolate the protective effects of antioxidant species from one species to another.

There is thus a need to develop a product for oral administration intended for a pet, based on the presence of antioxidants having demonstrated protective effects on pets, preferably on canidae or on felidae. In particular, there is a need for a product suitable for protecting or treating dogs predisposed to heart disease, particularly valve failure, such as small dogs. There is also a need to protect or treat older dogs, and large dogs suffering from other heart conditions.

Furthermore, it has also been demonstrated that a one-dimensional antioxidant use strategy was insufficient to obtain effective cardioprotective agents. There is thus a need to develop a product for oral administration intended for a pet, comprising a combination of substances including antioxidants and having complementary or inter-dependent effects.

It has already been demonstrated that L-carnitine and taurine, well known for the antioxidant activity thereof, can protect human endothelial cells from oxidative stress and can also have beneficial effects on some forms of canine cardiomyopathy (Sanderson, S. L. Taurine and carnitine in canine cardiomyopathy. Vet Clin North Am Small Anim Pract 36, 1325-43, vii-viii (2006)). These substances have become molecules of choice for formulation in dietary supplements for dogs suffering from cardiovascular problems.

Polyphenolic compounds, such as soy isoflavones obtained from soy extracts, and tannins extracted from pomegranate, have also been studied for the antioxidant activity thereof on cardiovascular disease in humans (Nicholson, S. K., Tucker G. A. & Brameld, J. M. Effects of dietary polyphenols on gene expression in human vascular endothelial cells. Proc Nutr Soc 67, 42-7 (2008)).

The document WO2010/002525 discloses a product for oral administration comprising punicalagins which are ellagitannins. They are particularly found in pomegranate peel and pomegranate juices. The product disclosed in the document WO2010/002525 further comprises L-carnitine and taurine, and may particularly be used to improve the cardiovascular health of pediatric subjects.

The documents EP2133080 and WO2010/012651 disclose products for oral administration comprising soy isoflavones, extracts of punica granatum (pomegranate), taurine and soy protein powder.

Furthermore, even though the antioxidant potential of these four substances has been evaluated individually in a plurality of in vitro tests, the ability thereof to protect animal endothelial cells, particularly canine endothelial cells remained unknown and uncertain to date.

### SUMMARY OF THE INVENTION

The solution to the problem addressed relates to a product for oral administration intended for a pet, comprising extracts of punica granatum (pomegranate), characterized in that it further comprises at least two additional antioxidant compounds. The additional antioxidant compounds are chosen from lemon bioflavonoids, vitamin C (ascorbic acid) or any of the potential derivatives/salts thereof such as calcium ascorbate, vitamin E, coenzyme Q10 (also known as ubiquinone), polyphenols, particularly resveratrol, maritime pine bark extract, green tea extracts, Gingko Biloba, lycopenes (for example tomato lycopene), taurine, carnitine, soy isoflavones. Preferably, these antioxidant compounds are chosen from soy isoflavone extracts, L-carnitine and taurine. Surprisingly, as described in examples 1 and 2, the applicant demonstrated that the association of extracts of punica granatum (pomegranate) with at least two further antioxidants is effective in the development of a multi-dimensional diet strategy for reducing the onset and progression of canine diseases associated with oxidative stress and protecting, treating or helping control the development of cardiovascular disease. The association of pomegranate extracts with L-carnitine, taurine and soy isoflavones is particularly effective.

The information for evaluating the cytoprotective effects of the substances was obtained using a test on canine aortic endothelial cells, as described in example 1. The effects of L-carnitine, taurine, extracts of pomegranate and soy isoflavones, used alone or in combination, were studied to determine the potential thereof in the development of a multi-dimensional diet strategy for reducing the onset and progression of canine diseases associated with oxidative stress.

The results of these studies demonstrate that pomegranate extracts, alone or combined with further substances, have a significant antioxidant and cytoprotective activity on canine endothelial cells.

The data resulting from this study particularly suggest that the four antioxidant substances, L-carnitine, taurine and extracts of pomegranate and soy isoflavones are extremely advantageous in the development of a multi-dimensional diet strategy for reducing the onset and progression of heart disease induced by oxidative stress in pets, more specifically for reducing the degeneration of endothelial cells involved in progressive valve failure.

The invention also relates to a product according to the invention as a complete food. It also relates to a product according to the invention as a supplementary or health food (nutritional supplements).

It also relates to a product according to the invention for treating or protecting cardiovascular disease. It also relates to a product according to the invention for helping control cardiovascular disease.

The aim of the invention is also that of providing a product for improving the health or living conditions of a pet, by administering an effective quantity of the product according to the invention.

The present invention further provides a method for reducing endothelial cell proliferation and/or apoptosis. The present invention provides a novel method for protecting endothelial cells from oxidative stress, in particular, for protecting canine endothelial cells. In this way, the present invention provides a product for supporting cardiac function in the case of chronic heart failure, particularly in dogs.

### BRIEF DESCRIPTION OF THE DRAWINGS

The invention will be understood more clearly on reading the following non-limiting description, with reference to the appended figures, wherein:

FIGS. 1 to 4 represent the results of studies intended to evaluate the effects of reference substances including antioxidants (Gluthathione, GSH and N-Acetylcysteine, NAC), vascular growth factors (Vascular Endothelial Growth Factor, VEGF; basic Fibroblast Growth Factor, bFGF), insulin receptor sensitizing agents (Pioglitazone and Rosiglitazone) on canine aortic endothelial cell proliferation and apoptosis;

FIGS. 5 to 8 show the results of studies evaluating the protective effect of natural substances against oxidative stress using a novel test developed on canine endothelial cells. In the case of FIG. 5, the effect on cell proliferation was evaluated. FIG. 6 shows the dose effect of pomegranate extract on cell proliferation. In FIG. 7, the effect on canine endothelial cell apoptosis is studied. FIG. 8 shows the dose effect of pomegranate extract on apoptosis.

FIGS. 9 and 10 show the results of studies intended to evaluate the protective effect of natural substances on cell death induced by oxidative stress. This study was considered using a novel test developed on canine endothelial cells and free radical reduction tests; and

FIG. 11 shows the results of a study intended to evaluate the antioxidant potential of natural substances by in vitro ABTS radical inhibition.

### DETAILED DESCRIPTION OF PARTICULAR EMBODIMENTS

The product for oral administration intended for a pet according to the invention comprises extracts of punica granatum (pomegranate), and at least two additional antioxidant compounds chosen from:
soy isoflavone extracts;
L-carnitine; and
taurine.

According to one embodiment of the invention, the product for oral administration intended for a pet comprises:
extracts of punica granatum (pomegranate);
soy isoflavone extracts; and
L-carnitine.

According to a further embodiment of the invention, the product for oral administration intended for a pet comprises:
extracts of punica granatum (pomegranate);
soy isoflavone extracts; and
taurine.

According to one alternative embodiment of the invention, the product for oral administration intended for a pet comprises:
extracts of punica granatum (pomegranate);
L-carnitine; and
taurine.

According to one particularly preferred embodiment of the invention, the product for oral administration intended for a pet comprises:
extracts of punica granatum (pomegranate);
soy isoflavone extracts;
L-carnitine; and
taurine.

According to one embodiment of the invention, the product comprises:
between 1% and 90%, between 1% and 80%, between 1% and 50%, between 5% and 20% by weight of the total weight of the product of L-carnitine;
between 1% and 90%, between 1% and 80%, between 1% and 50%, between 5% and 30% by weight of the total weight of the product of taurine;
between 0.1% and 90%, between 0.1% and 50%, between 0.1% and 10%, between 0.1% and 5% by weight of the total weight of the product of extracts of punica granatum (pomegranate); and
between 0.1% and 90%, between 0.1% and 50%, between 0.1% and 10%, between 0.1% and 5% by weight of the total weight of the product of concentrated soy isoflavone extracts.

According to one preferred embodiment of the invention, the product comprises:
between 5% and 15% by weight of the total weight of the product of L-carnitine;
between 10% and 20% by weight of the total weight of the product of taurine;
between 0.1% and 5% by weight of the total weight of the product of extracts of punica granatum (pomegranate); and
between 0.5% and 5% by weight of the total weight of the product of concentrated soy isoflavone extracts.

However, the choice and precise quantity of each additional ingredient is determined by the end use of the product according to the invention. This choice is particularly dependent on one or more parameters such as the type of food wherein these ingredients are to be added (complete food, supplementary food, health food), the galenic form of the finished product (capsule, tablet, powder, solution, etc.), the species, age, body weight, general health, sex, consumption rate, type of disease or condition treated, of the animal for which the product according to the invention is intended. Consequently, the quantities of the additional ingredients may vary considerably.

The product according to the invention may be in the form of a complete food or a supplementary or health food.

The product according to the invention is useful for treating or protecting cardiovascular disease, for helping control cardiovascular disease, or for supporting cardiac function in the case of chronic heart failure.

The product according to the invention is particularly suitable for administration to a canid or a felid, particularly a canid selected from dogs of the following breeds: Poodle, Chihuahua, Bichon, Yorkshire, King Charles Spaniel, Pekinese, Pinscher, Keeshond, Spaniels, English Springer Spaniel, Pomeranian, Basset, Beagle, Westie, Whippet, Terriers, Fox Terrier, Yorkshire Terrier.

In a further aspect, the product according to the invention is administered daily for a period of at least 1, 2, 3, 4, 5 or 6 months. In a further aspect of the invention, the product is administered at a dose between 0.0001 mg/kg and 350 mg/kg. In a further aspect of the invention, the product is in the form of a tablet, capsule, sugar-coated tablet, powder, granule, syrup, suspension, solution or emulsion.

Pomegranate is the fruit of the pomegranate tree (punica granatum) of the Lythraceae family. Pomegranate extract has strong antioxidant and anti-inflammatory potential associated with the presence of anthocyans, ellagic tannins and hydrolysable tannins. Pomegranate also has a protective and vasorelaxant effect on the endothelium. Punica granatum (pomegranate) may be more or less concentrated in punicosides and antioxidants according to the source of pomegranate used, as discussed in example 2, pomegranate sources comprising a high proportion of punicosides are preferred, in particular, pomegranate extracts comprising more than 10%, 20%, 30% or 40% punicosides.

Soy isoflavones are molecules of the flavonoid family having estrogen-like properties. Genistein and Daidzein are the main soy flavonoids. Soy isoflavones are antioxidants having a protective and vasorelaxant effect on the endothelium. Genistein is an antioxidant capable of neutralizing the negative effects of free radicals on endothelial cells forming blood vessels.

Carnitine is a compound comprising a quaternary ammonium function, it is biosynthesized from lysine and methionine. This molecule is involved in the cells in cytosol fatty acid transport to the mitochondria during the lipid catabolism in the energy metabolism. Carnitine has two stereo-isomers, the active biological form thereof is L-carnitine whereas the D form is biologically inactive. L-carnitine is an antioxidant also having anti-arrhythmic properties.

Taurine is derived from a sulfur-containing amino acid: cysteine. Taurine is naturally present and synthesized in the body. It is an antioxidant involved in the energy metabolism, in cardiac and muscle function, particularly by increasing cardiac contractility, i.e. inotropism.

The product according to the invention is in any galenic form normally used for oral, i.e. per os, administration. The product according to the invention is advantageously in solid form or in liquid form.

The product according to the invention may be in the form of a tablet, capsule, sugar-coated tablet, powder, granule, or in the form of syrup, suspension, solution, emulsion, pre-concentrate or suspension of microspheres or nanospheres or lipid or polymeric vesicles suitable for controlled release. It may also be in a form incorporated in a finished product in the form of a gel, paste, biscuit, strip or a toy for chewing such as for example an artificial bone. The product according to the invention is incorporated at a content between 0.001% and 90%, between 0.001% and 50%, between 0.001% and 20%, between 0.01% and 20%, or between 0.01% and 10% in total weight of the finished product. The quantity of product according to the invention in such finished products may be determined by those skilled in the art according to the end use of the product, the animal to be treated, the product form.

It may further be in the form of a fluid mixture to be added to or into a food composition. According to the embodiment of the invention, the product according to the invention may a nutritional value or not. In this way, the invention also relates to a food, which is for example a complete food or a supplementary food (supplementary or health food), for pets, for example for canidae, comprising the constituents of the product according to the invention.

Advantageously, the product according to the invention also comprises one or a plurality of additional ingredients well known to those skilled in the art such as in particular feeding agents, binding agents, granulation agents, lubricants, flavors, colorants, fillers, sweeteners, emulsifiers, flavor enhancers, minerals, film-coating agents, salts, stabilizers, buffers or vitamins. Stabilizers include substances which tend to increase the shelf-life of the composition such as preservatives, emulsifiers, thickeners, packing gases, gelling agents, humectants, sequestering agents, synergists or stabilizers. As binding agents, mention may be made for example of polyvinyl alcohol, polyvinylpyrrolidone polymers, vinylpyrrolidone and vinyl acetate copolymers, polyvinyl acetophthalate, celluloses and derivatives thereof, alginic acid and salts thereof, zein, hyaluronic acid, pectins, gum arabic, tragacanth, gum karaya, xanthan gum, carrageenans, pullulan or agar polymers, chitosan and derivatives thereof, carbomers, cross-linked acrylic acid with polyalkenyl ethers, polycarbophils, methylvinyl and maleic anhydride copolymers, non-ionic block copolymers of polyoxyethylene and polyoxypropylene, monosaccharides, disaccharides and polysaccharides, polyols and mixtures thereof. The term celluloses and derivatives thereof particularly denotes monocrystalline cellulose, alkyl cellulose ethers or esters such as methyl cellulose, ethylcellulose, cellulose acetophthalate, cellulose acetate. As diluents, mention may be made for example of polyvinyl alcohol, polyvinylpyrrolidone polymers, vinylpyrrolidone and vinyl acetate copolymers, polyvinyl acetophthalate, celluloses and derivatives thereof, alginic acid and salts thereof, zein, hyaluronic acid, pectins, gum arabic, tragacanth, gum karaya, xanthan gum, carrageenans, pullulan or agar polymers, chitosan and derivatives thereof, carbomers, cross-linked acrylic acid with polyalkenyl ethers, polycarbophils, methylvinyl and maleic anhydride copolymers, non-ionic block copolymers of polyoxyethylene and polyoxypropylene, monosaccharides, disaccharides and polysaccharides, polyols and mixtures thereof, sugars such as glucose, maltodextrins, sorbitol, sucrose.

A feeding agent suitable for the target species, particularly domestic carnivores such as dogs and cats, will be chosen, for example brewer's yeast, meat meals, fish meals, powdered cheese or milk derivatives, powdered liver and mixtures thereof, amino acids and mixtures thereof, natural or artificial flavors.

As granulation agents, mention may be made for example of methacrylate copolymers, ethylcellulose and derivatives thereof. In one particular embodiment of the invention, the cationic copolymer of dimethylaminoethyl methacrylate, butyl methacrylate, and methyl methacrylate (known under the trade name Eudragit E100®) or any derivative thereof, which may give the composition enhanced moisture resistance properties, will be chosen.

As lubricants, mention may be made for example of stearic acid and derivatives thereof, citric acid and derivatives thereof, lactic acid and derivatives thereof, propylene glycols, glycerin, phthalates and derivatives thereof, adipates and derivatives thereof, sebacates and derivatives thereof, polyethylene glycols and derivatives thereof, glyceryl behenate.

As flavor enhancers, mention may be made of sucrose and sugar derivatives such as sucrose, sucralose.

In one alternative embodiment of the invention, further agents may also be added for the beneficial properties thereof on the cardiac and vascular system (inotropic, anti-arrhythmic, hypotensive, vasodilator effects, etc.), such as: folic acid or vitamin B9, omega-3 fatty acids, metals such as selenium or magnesium, Crataegus oxycantha extract, L-arginine.

The product according to the invention is intended for a pet, i.e. an animal that has been domesticated. Preferably, the pet is chosen from mammals (felidae, canidae, equidae, lagomorphs, rodents). Advantageously, the product according to the invention is intended for dogs and cats, however, the term pet also includes new pets such as for example weasels, rodents (for example rabbits, hamsters), miniature pigs.

In one aspect of the invention, the product according to the invention is intended for dogs. The product and the methods according to the invention are more particularly intended for small dogs or breed dogs having a predisposition to heart problems. Such dog breeds are for example and not exhaustively: Poodle, Chihuahua, Bichon, Yorkshire, King Charles Spaniel, Pekinese, Pinscher, Keeshond, Spaniels, English Springer Spaniel, Pomeranian, Basset, Beagle, Westie, Whippet, Terriers, Fox Terrier, Yorkshire Terrier. The term “small dog” denotes, within the scope of the present invention, dogs of less than 15 or 10 kilos.

In a further aspect of the invention, the dog is a large dog. The term “large dog” denotes, within the scope of the present invention, dogs of more than 30 kilos. For example, and non-exhaustively, a breed dog chosen from: Newfoundland, Bobtail, Labrador, German Shepherd, Collie, Doberman, Boxer.

Dogs which are not purebred but have similar features to said breeds may also be treated using the product according to the present invention.

According to one embodiment of the invention, the product according to the invention may be administered in combination with medicinal products usually prescribed for animals suffering from heart conditions, such as for example aldosterone antagonists, inodilators, converting enzyme inhibitors, angiotensin comprising inter alia: Benazepril, Captopril, Cilazapril, Enalapril, Fosinopril, Imidapril, Lisinopril, Moexipril, Perindopril, Quinapril, Ramipril, Spirapril, Trandolapril.

The product according to the invention is, according to a further embodiment, a complete food, a supplementary or health food, i.e. a product combining the concept of food and medication in the context of functional nutrition. It thus makes it possible by definition to reduce the risks or protect the onset of some diseases, using substances contained in a staple food. The term supplementary or health food is used herein to denote a food which has a positive impact on an animal's health and/or physical performances, in addition to the basic function of providing nutrients. According to the invention, the benefits observed with the product particularly relate to cardiovascular disease.

The food according to the invention may thus comprise substances other than the additional ingredients detailed above, particularly plant extracts, dietary raw materials (vegetable, animal, mineral), amino acids, proteins, essential oils, fatty substances (fatty acids in particular), minerals, vitamins or medicinal active substances.

As detailed in the examples hereinafter, according to the invention, the product is advantageously used for treating, protecting or helping control cardiovascular disease. Within the scope of the present invention, the term “helping control cardiovascular disease” denotes a means for supporting cardiac function in animals, particularly those predisposed to developing cardiovascular disease. Preferably, the product according to the invention is suitable for supporting cardiovascular function in dogs and particularly those suffering from or predisposed to developing valve disease, particularly mitral valve disease, small dogs, said product being intended to be given to dogs, particularly older dogs, every day and on a long-term basis.

In one alternative embodiment of the invention, the product may be administered daily, throughout the animal's lifetime. In a further alternative embodiment, the product may also be administered regularly 1, 2, 3 times a day or every 2, 3, 4 days, 1, 2, 3 times a week or 1, 2, 3, 4 times a month.

In a further alternative embodiment, the product may also be administered in the form of courses or cycles of treatment. This means that the product is administered, daily or not, over a defined period of time, between 1 week and one year, more particularly for 1, 2, 3, 4, 5 or 6 months. In one alternative embodiment of the invention, the product according to the invention is administered at least 2, 3, 4, 5, 6 or several times to the animal. In one embodiment, the product is administered in the form of a course of 1, 2, 3, 4, 5, 6 or several months, 1, 2, 3 times a year. In one particular alternative embodiment of the invention, the product is administered daily, for a period between 1 and 6 months, in the form of a repeatable course once to twice a year.

In a further particular alternative embodiment, the product is administered daily for a period of at least 1, 2, 3, 4, 5 or 6 months.

The product according to the invention is administered in a dose and at a frequency which may be chosen and adjusted by those skilled in the art. As a general rule, the product according to the invention is administered at a dose between 0.0001 mg/kg and 350 mg/kg, between 0.001 mg/kg and 350 mg/kg, between 0.01 mg/kg and 350 mg/kg, between 1 mg/kg and 350 mg/kg, between 10 mg/kg and 350 mg/kg. The total quantity of pomegranate extract contained in the product according to the invention varies preferentially from 0.001 mg/kg to 350 mg/kg, preferably from 0.01 mg/kg to 50 mg/kg, more preferably from 0.1 mg/kg to 35 mg/kg.

Alternatively, when the product according to the invention is incorporated in a complete food, the daily dose may be expressed in milligrams per calorie unit. The product according to the invention is administered in a quantity between 0.01 mg/500 kcal and 350 mg/500 kcal, between 1 mg/500 kcal and 350 mg/500 kcal, between 10 mg/500 kcal and 350 mg/500 kcal. The total quantity of pomegranate extract contained in the product according to the invention may thus vary from 0.001 mg/500 kcal to 350 mg/500 kcal, preferably from 0.01 mg/500 kcal to 50 mg/500 kcal, preferably from 0.1 mg/500 kcal to 35 mg/500 kcal.

The invention also relates to a method for treating or protecting heart disease in animals, comprising the administration of a composition according to the invention, in a sufficient quantity to treat or protect heart disease. The sufficient quantity is at least 0.0001 mg/kg, at least 0.001 mg/kg, at least 0.01 mg/kg. The treatment is administered over a period of at least 1, 2, 3 weeks or 1, 2, 3, 4, 5 or 6 months.

In the context of the invention, the doses cited denote daily doses in the case of a daily treatment, These doses may also be administered daily only for a given period of time, for a course of treatment for example.

In one aspect of the invention, the product is used for non-therapeutic purposes, for example for improving the general state of the animal. Indeed, the product according to the invention may have an energizing effect, an effect on reducing cell ageing and, consequently, reduce the incidence of diseases and promote the recovery of the animal after an illness or accident, or on increasing vitality. These benefits will be visible through an improvement in the visual appearance of the animal (shiny coat, improved posture), the tonicity thereof, through the improvement in the behavior thereof (interaction with master, sleep, appetite, playfulness), the improvement in the general state will also prevent the onset of opportunistic diseases.

### Example 1

### In Vitro Effects of Vasoprotector Agents in Canine Aortic Endothelial Cells

Using CnAOEC cells from Cell Applications, Inc, a cell culture model was established, suitable for testing essential cell functions, such as proliferation and apoptosis, in response to various stimuli. The system established is suitable for identifying pro- and anti-proliferation stimuli, and pro- and anti-apoptotic stimuli.

Antioxidants being the phase 2 test substances of the study, it is important that the tests set up enable the detection of the anti-proliferation potential and the anti-apoptotic effects previously observed in endothelial cells exposed to the following antioxidants: glutathione (GSH) and N-acetylcysteine (NAC). The reference anti-proliferation and anti-apoptotic activities of these substances displayed considerable reproducibility throughout the study.

A. Phase 1 for Setting Up the Pro/Anti-Proliferation and Pro/Anti-Apoptotic Effect Potential Evaluation Tests:

The CnAOEC cells were cultured in culture dishes coated with a specific culture medium. The CnAOEC cells were labeled with ³H-thymidine and exposed to reference agents known to modulate apoptosis and proliferation in various types of human vascular endothelial cells. These reference substance include antioxidants (Glutathione and N-acetylcysteine), free fatty acids (linoleic acid (ALenS) and γ-linolenic acid (Lols))), vascular growth factors (Vascular Endothelial Growth Factor (VEGF) and basic Fibroblast Growth Factor (bFGF)) and insulin receptor sensitizing agents (Pioglitazone and Rosiglitazone). After incubating the cells with test and reference substances, proliferation and apoptosis tests were performed as described in more detail hereinafter.

To set up the tests with ³H-thymidine evaluating proliferation and apoptosis in CnAOEC cells, various aspects were evaluated.

a. Characterization of Canine Aortic Endothelial Cells and the Method for the Culture Thereof: Experimental Conditions for Inoculation (Number of Cells Required, Cell Detachment and Attachment Conditions, Growth Rate, Etc.)

The cells are cultured in a culture medium containing CECGM (Canine Endothelial Cell Growth Medium) growth factors and previously coated with AFS (Attachment Factor Solution) agent to assist with the adherence thereof. For the development of the test, the cells were inoculated at a density of 5000 to 10,000 cells/cm² in culture dishes pretreated with fibronectin (0.0025% in a PBS buffer).

b. Proliferation Tests:

The confluent CnAOEC cell cultures were re-inoculated in 96-well culture plates (10,000 cells per plate). Six hours after the inoculation thereof, the cells were exposed to ³H-thymidine (final concentration: 1 μCi/ml) and to the respective test agents for 48 hours, and were subjected to two washing steps with PBS. After a treatment with trypsin and a freeze-thaw cycle, the DNA incorporating ³H-thymidine was captured on a glass fiber filter using a “Betaplate™ 96-bien-Cell Harvester” (Wallac, Turku).

The quantities of ³H-thymidine incorporated in the control cells (without adding test agent) were defined as 100% and the results were expressed as a function of these control cells.

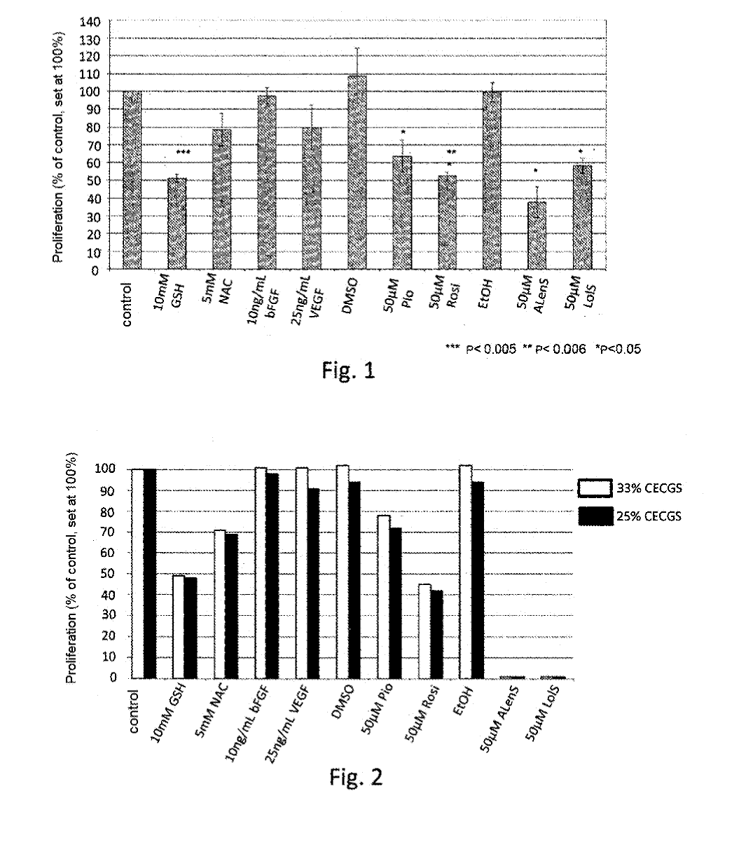

As shown in FIG. 1 illustrating the effects of the various reference substances on CnAOEC cell proliferation (mean of three independent experiments, performed in four wells), the antioxidant glutathione (GSH), the insulin sensitizing agents Pioglitazone (Pio) and Rosiglitazone (Rosi), and free fatty acids (linoleic (ALenS) and γ-linolenic (Lols) acids) reduce CnAOEC cell proliferation considerably. On the other hand and surprisingly, vascular endothelial growth factor (VEGF) and fibroblast growth factor (bFGF), known to increase human endothelial cell proliferation, did not induce the same response in CnAOEC cells.

As shown in FIG. 2 illustrating CnAOEC cell proliferation, in a culture medium with reduced CECGS, although proliferation is almost completely inhibited (<2%) by the two free fatty acids, the anti-proliferation effects remain within a similar range for vascular endothelial growth factor (bFGF), basic fibroblast growth factor (VEGF), glutathione (GSH), Rosiglitazone (Rosi) and Pioglitazone (Pio). In this way, it is deduced from these experiments that bFGF and VEGF, unlike human endothelial cell culture models, have no influence on CnAOEC cell proliferation. The same proportions of DMSO (1%) and ethanol (1.5%) are used as solvents for free fatty acids or for further test substances.

c. Tests of Reference Substances on Apoptosis in CnAEOC Cells:

The 60 mm plates containing semi-confluent CnAOEC cells were labeled with ³H-thymidine (1 μCi/ml) for 36 hours. These cells were then inoculated in 24-well culture plates (50,000 cells/well). After the exposure thereof to the agents under test (24 hours), the cells were treated with a lysis buffer (20 mmol/1 TrisHCl, pH 7.5, and 0.4% of Triton X-100 in PBS). The radiolabeled fragmented (apoptotic) DNA present in the supernatant was counted using a liquid scintillation analyzer. The results were then correlated with the total radioactivity incorporated in the cells (quantified after digestion of the remaining suspension with 180 μg/ml of DNase). The values calculated for the control cells (without adding test agent) were set at 100% and the results were expressed as a function of these control cells. Each time the reference substances were dissolved in ethanol (final concentration 1.5%), an identical quantity of ethanol was added to all the other wells, including the control wells.

d. Apoptosis Tests

The apoptosis tests were conducted with radiolabeled CnAOEC cells, at 60% confluence and in culture dishes previously treated with fibronectin. At this confluence, cell apoptosis is inhibited after the exposure thereof for the antioxidants glutathione and N-acetylcysteine. On the other hand, exposing the cells to free fatty acids (linoleic acid and γ-linolenic acid) caused a very strong pro-apoptotic reaction.

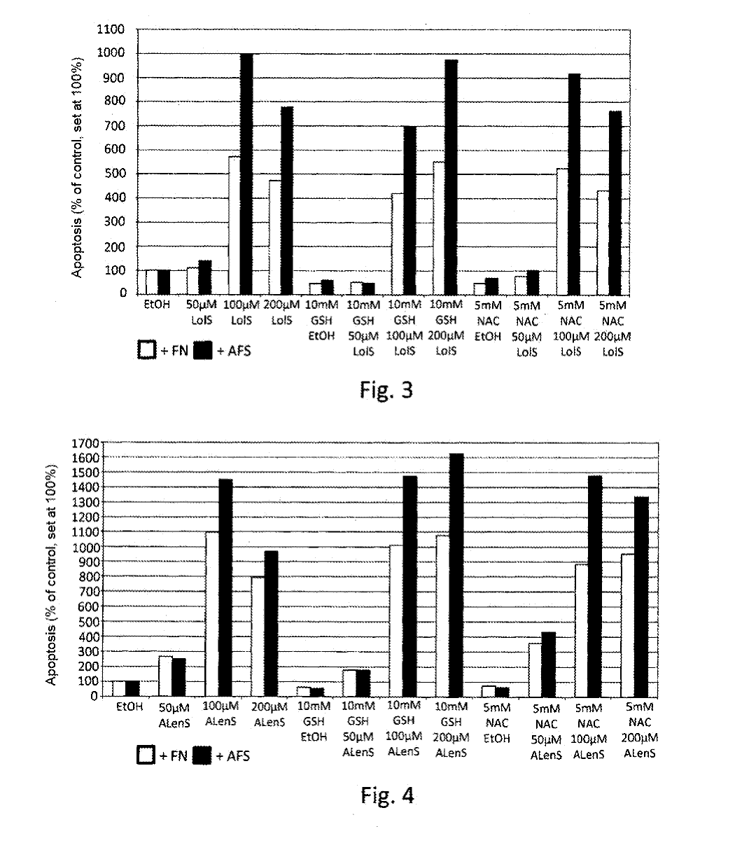

25 μM of free fatty acids weakly inducing apoptosis in CnAOEC cells, the concentrations to be used for the free fatty acids were thus set at 50 μM, 100 μM and 200 μM for subsequent experiments. Furthermore, as shown in FIGS. 3 and 4 illustrating CnAOEC cell apoptosis, the pro-apoptotic effects of free fatty acids are not completely inhibited by the antioxidants glutathione (GSH) and N-acetylcysteine (NAC), either for linoleic acid (ALenS) or for γ-linolenic acid (LolS).

Conclusion for Phase 1:

In the first phase of the study, protocols for CnAOEC cell culture (in culture dishes coated with fibronectin and in culture medium for canine endothelial cells) and for performing the proliferation and apoptosis tests were drafted.

The glutathione and N-acetylcysteine and the insulin sensitizing agents Pioglitazone and Rosiglitazone reduce cell proliferation by 30% to 70%, whereas free fatty acids (linoleic acid and γ-linolenic acid) inhibit endothelial proliferation completely. Unlike the effect thereof on human vascular endothelial cells, bFGF and VEGF have no pro-proliferation effect on CnAOEC cells.

The antioxidants glutathione and N-acetylcysteine inhibit apoptosis in CnAOEC cells significantly, whereas free fatty acids cause a strong pro-apoptotic reaction. These data demonstrate that the design of the experiment set up is suitable for testing substances both for pro- and anti-apoptotic effects.

B. Phase 2 for Evaluating the Substances Under Test.

a. Evaluation of Substances Under Test on CnAOEC Cell Proliferation:

Before each experiment, a new stock solution of each of the substances under test (taurine, carnitine, extracts of pomegranate and soy isoflavones) was prepared. The soy isoflavones were solubilized in DMSO. The experimental samples, and those of the respective controls (DMSO free from test substance) contained a final DMSO concentration of 1%. The substances: taurine, L-carnitine, soy isoflavone concentrate and pomegranate extract were tested alone or in association with a final concentration of 50 μg/ml in the culture medium. The results obtained were compared to those obtained for the control wells (free from test substances, but with the same concentration of solvent, for example DMSO).

For the mixture of the 4 test substances, the concentration is 250 μg/ml, i.e. 62.5 μg/ml of each test substance.

The final DMSO concentration in the control and in the experimental samples exposed to the mixture was adjusted to 1%.

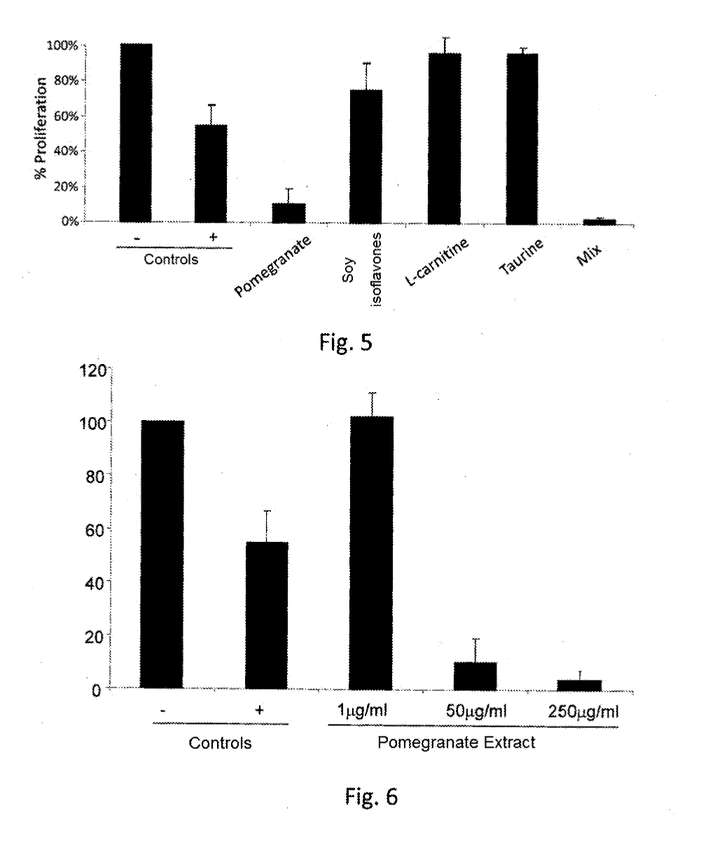

As illustrated in FIG. 5, the substance taurine (Tau), 40% pomegranate extract (Pom), L-carnitine-tartrate (Car), soy isoflavones (Soy) (40% isoflavones) and a mixture of these substances (Mix) were tested using the 50 μg/ml concentrations for the substances alone and 62.5 μg/ml per substance for the mixture (i.e. 250 μg/ml) compared to concentrations of 10 mmol/1 for the glutathione (GSH) control for the efficacy thereof in CnAOEC cell proliferation.

The statistical evaluation is based on three or four separate experiments, each being performed with 2 to 4 independent wells.

Carnitine (Car) and Taurine (Tau) did not clearly modulate CnAOEC cell proliferation. For these two substances, merely a slight but significant reduction in proliferation is observed with a 3.75% drop for carnitine (Car; 96.25% renewal rate) and 4% for taurine (Tau; 96% renewal rate).

In respect of pomegranate and soy isoflavones, significant inhibition of cell proliferation is observed with renewal rates which are merely in the region of 10.5% for pomegranate extract and 75% for soy isoflavone concentration compared to the positive control (set at 100%). Interestingly, the positive control used, glutathione, reduced the proliferation to merely 54.75% of the control.

FIG. 5 also shows that the use of a mixture of the substances (Mix) reduces cell proliferation considerably to 2.75% of the control (set at 100%). This significant inhibitory effect is potentially due to the anti-proliferation action of pomegranate (Pom) and soy isoflavones (Soy), even if in the mixture, only 25% of each of the four substances was present. Therefore, a synergistic effect of at least two compounds of the mixture of the 4 substances (Mix) is observed inducing a considerable anti-proliferation action on CnAOEC cells.

FIG. 6 shows, under the same experimental conditions, the dose effect results (1 μg/ml, 50 μg/ml and 250 μg/ml) for pomegranate alone. This FIG. 6 demonstrates that the effect on cell proliferation is obtained in a dose-dependent manner with pomegranate alone.

b. Evaluation of Test Substances on CnAOEC Cell Apoptosis:

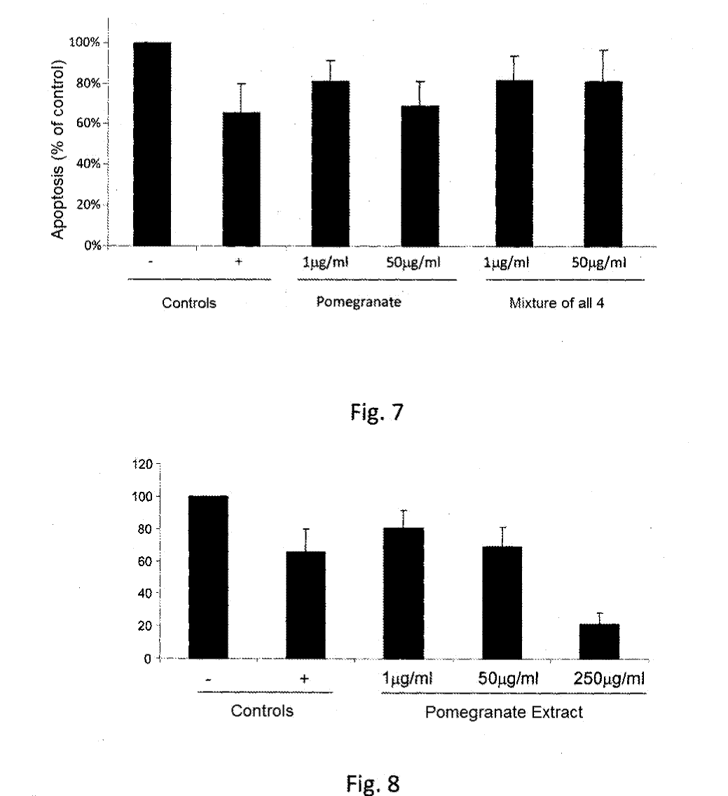

As illustrated in FIG. 7, 40% pomegranate extract (Pomegranate) and a mixture of the substances taurine, pomegranate extract, L-carnitine-tartrate, soy isoflavones (mixture of the 4) were tested using the 1 and 50 μg/ml concentrations compared to 10 mmol/1 of the antioxidant glutathione (GSH). FIG. 8 shows a dose response of the effect of pomegranate extract alone at the 1 μg/ml, 50 μg/ml and 250 μg/ml doses.

The statistical evaluation is based on three separate experiments, each being performed with 2 to 4 independent wells.

The modulation of CnAOEC cell apoptosis (variation measured as a % relative to the control set at 100%) is performed in comparison to the reference substance glutathione (GSH).

Pomegranate extract (particularly containing 40% punicosides) significantly reduces apoptosis at all the concentrations tested, in a dose-dependent manner. In this way, at a concentration of 250 μg/ml, a 74% reduction compared to the control cells (set at 100%) is observed.

The mixture of the 4 substances (Mix), as illustrated in FIG. 7, induces a significant reduction of apoptosis compared to the control cells (solvent free from test substances). Nevertheless, these reductions are not extensive enough to be statistically significant.

Conclusion for Phase 2:

As mentioned above, extracts of pomegranate and soy isoflavone reduce proliferation and apoptosis in a dose-dependent manner, pomegranate extract having greater effects.

It is interesting to note that, at the concentrations tested, pomegranate extract has greater anti-proliferation and anti-apoptotic effects than the antioxidant glutathione. The mixture appears to reduce proliferation considerably at a concentration of 250 μg/ml, whereas for apoptosis, the mean values are less than those of the respective controls thereof.

### Example 2

### Evaluation of the Protective Effect of Natural Substances Against Oxidative Stress Using a Novel Test Developed on Canine Endothelial Cells and Radical Scavenging Assays

The aim of this example is that of evaluating the protection against oxidative stress induced by H₂O₂ in canine endothelial cells and the free radical reduction potential using antioxidant natural substances. Before these studies, no compound had been described for an in vitro effect against oxidative stress in canine endothelial cells. Furthermore, according to Ram & Hiebert (see above), the protective effect observed on aortic endothelial cells with natural antioxidant molecules could be species-dependent.

In the experiments conducted hereinafter, the various chemical products and cell culture reagents were obtained as follows:
canine aortic endothelial cells (CnAOEC) and canine endothelial cell growth medium (CECGM) are supplied by Tebu-Bio™;
the products Trolox™, ABTS (2,2′-azino-bis(3-ethylbenzthiazoline-6-sulfonic acid), H₂O₂, MTT (3-(4,5-dimethylthiazol-2-yl)-2,5-diphenyltetrazolium bromide) are supplied by Sigma-Aldrich™;
the soy isoflavone concentrate is 40% concentrated with isoflavones and supplied by ADM™;
the pomegranate extract concentrated with 40% punicosides is supplied by Polynat; and finally
L-carnitine-L-tartrate and taurine are supplied by Arnaud SAG™.

Bioavailability data are available for dogs for taurine, carnitine and genistein (one of the main soy isoflavone aglycones). These data demonstrate that plasma concentrations after oral supplementation may be up to 50 μg/ml for taurine and carnitine and 5 μg/ml for genistein.

In respect of pomegranate extract, data are only available for humans. It was demonstrated in these studies that ellagic acid, one of the main polyphenols of dietary pomegranate, can be found in the plasma at concentrations of approximately 10 μg/ml after dietary supplementation with pomegranate extract.

The CnAOEC cells were thawed and cultured in canine endothelial cell growth medium with the supplier's instructions.

The cells were inoculated at various densities of 2.10⁴ to 5.10⁴ cells/cm² in 96-well plates for monitoring the behavior thereof in culture. At 5.10⁴ cells/cm², the cells present the typical appearance thereof and achieved 100% confluence after 7 days of culture. The behavior and appearance of the CnAOEC cells remain unchanged between passages 1 and 14, these cells were used up to passage 14 at most.

In this example, the experimental data from the acellular systems are expressed as a mean±SD (standard deviation) and analyzed using Student's test with commercial software (SYSTAT, SPSS Inc, Chicago, Ill.).

The data obtained with the natural substances in the test based on CnAOEC cells were analyzed statistically by means of a one-way ANOVA. The differences were considered to be significant at p<0.05.

a. Measurement of Cell Viability Using an MTT Test:

The MTT test was used for evaluating the viability of CNAOEC cells exposed to cytotoxic treatments (60 μM of digitonin or addition between 0.1% and 20% of DMSO or at increasing concentrations (ranging from 0.5 mM to 16 mM) of H₂O₂ for 24 hours so as to induce oxidative stress.

In this test, the cell damage is evaluated 24 hours after treatment with the cytotoxic substance by means of a 4-hour incubation with MTT at 0.5 mg/ml, 37° C. and 5% CO₂. The plates are then centrifuged, and the formazan resulting from the reaction with MTT is solubilized in DMSO. The absorbance is measured at 570 nm, with a correction at 690 nm.

The MTT test was chosen to measure the cell viability on the basis of the data obtained after treating the cell with 60 μm digitonin (73% reduction±10%) and with 10% and 20% DMSO.

The damage to the CnAOEC cells caused by free radicals is obtained from a treatment with H₂O₂ at a concentration range from 0.5 mM to 16 mM.

The results of the MTT analysis indicate that the cell viability is significantly diminished after 24 hours of exposure.

These conditions were chosen for the purposes of screening the natural substances selected.

b. Evaluation of Potential of Natural Substances for Protecting CnAOEC Cells Against Oxidative Damage:

The ability of the natural substances to protect cells against the adverse effects of oxidative stress was evaluated in CnAOEC cells. The various samples were diluted in culture medium and no solubilization problem was observed with each of the substances tested. The cells were then pretreated for 8 hours with increasing concentrations (1 μg/ml, 50 μg/ml, 250 μg/ml) of soy isoflavone extract (Soy), pomegranate extract (Pom), L-carnitine (Car), taurine (Tau) and the SPCT mixture (Mix), i.e. the mixture of the 4 substances wherein each of the substances is present respectively at 0.25 μg/ml, 12.5 μg/ml and 62.5 μg/ml in order to obtain the same concentrations in the final sample thus 1 μg/ml, 50 μg/ml, 250 μg/ml. After the pretreatment period, the cells were subjected to oxidative stress (2 mM H₂O₂) for 24 hours and the cell viability was evaluated using the MTT test as described above.

For this test, the data are means obtained from eight different experiments±Standard Deviation (SD). They were analyzed statistically using a one-way ANOVA Student t-test. The differences were considered to be significant when p<0.05.

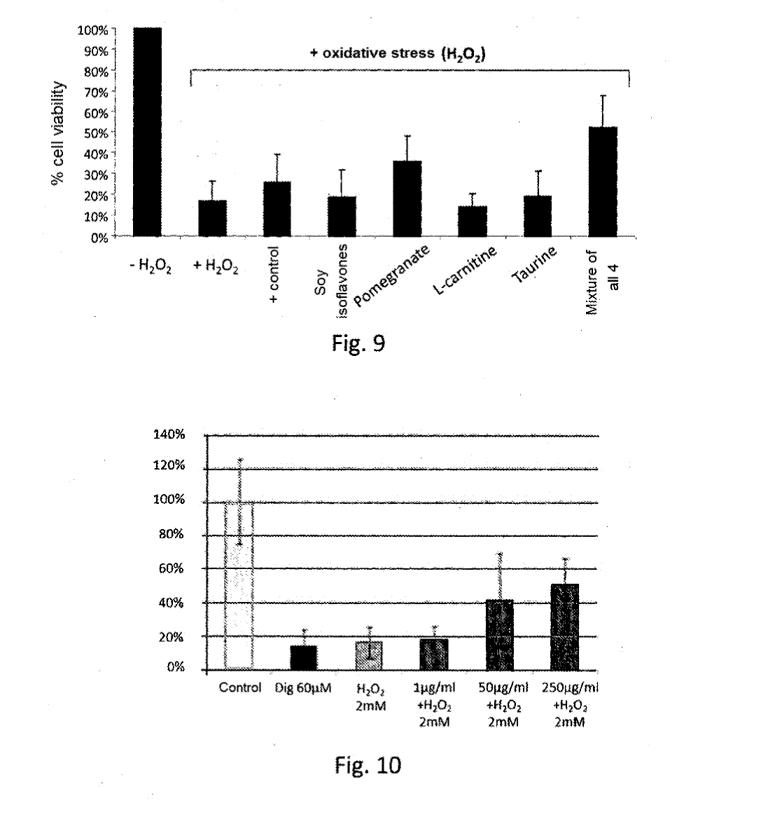

The protective effect of the pretreatment with natural substances on the cell viability of cells treated with H₂O₂ was evaluated using the MTT test. The data demonstrated that, as illustrated in FIG. 9, at the 50 μg/ml dose, taurine, L-carnitine, soy isoflavone extract, are not capable of maintaining CnAOEC cell viability during the application of oxidative stress at 2 mM of H₂O₂.

However, as illustrated in FIG. 9 at the 50 μg/ml dose and in FIG. 10, for the 1 μg/ml and 250 μg/ml concentrations, pomegranate extract displays a strong and significant protective effect on CnAOEC cell viability against oxidative damage induced by H₂O₂, in a dose-dependent manner. Indeed, at 50 μg/ml and 250 μg/ml, pomegranate extract improves cell viability following oxidative stress by 35% and 82% respectively. Furthermore, this protective effect is observed further when pomegranate extract is mixed with other substances. Indeed, as illustrated in FIG. 9, the combination of the 4 substances at 250 μg/ml (corresponding respectively to a 62.5 μg/ml concentration for each ingredient) improves CnAOEC cell viability by 51%.

The level of cell protection induced by mixing the four substances is comparable to the protection induced by pomegranate extract alone.

c. Trolox Equivalent Antioxidant Capacity (TEAC) Assay:

This method is based on the ability of antioxidant molecules to inactivate ABTS° ⁺ radical with a long lifetime, compared to Trolox, a water-soluble vitamin E analog, used as the positive control. ABTS° ⁺ radical is blue-green chromophore with a characteristic absorption at 734 nm.

The samples of the test substances and Trolox are diluted in PBS buffer to obtain the chosen concentrations, Then, 200 μl of ABTS° ⁺ at a concentration of 7 mM is added to 50 μl of sample or Trolox solution in 96-well plates.

The absorbance of the various samples is read every 2 minutes for 6 minutes (incubation time) at ambient temperature. The results are given as a percentage of ABTS radical inhibition, and as Trolox equivalent (μM).

The TEAC test results are reproduced in table A hereinafter:

TABLE A

TEAC test
ABTS° + inhibition

| | | |
|---|---|---|
| | | Standard |
| Sample | % | deviation |

| | | | |
|---|---|---|---|
| Control | | 0.00 | 4.09 |
| Soy isoflavones (μg/ml) | 1 | 9.04 | 1.94 |
| | 50 | 83.44 | 5.42 |
| | 250 | 91.62 | 2.99 |
| Pomegranate (μg/ml) | 1 | 27.84 | 4.12 |
| | 50 | 97.37 | 2.75 |
| | 250 | 97.38 | 2.61 |
| L-carnitine (μg/ml) | 1 | 0.45 | 4.95 |
| | 50 | 0.37 | 4.59 |
| | 250 | 0.92 | 4.19 |
| Taurine (μg/ml) | 1 | −0.50 | 4.02 |
| | 50 | −0.69 | 4.42 |
| | 250 | −0.62 | 4.66 |
| SPLT mixture (μg/ml) | 1 | 8.91 | 3.57 |
| | 50 | 97.16 | 2.66 |
| | 250 | 96.79 | 2.82 |

As demonstrated in table A above, the extracts of soy isoflavones and pomegranate displayed a strong antioxidant activity. Indeed, the pomegranate extract almost completely quenched, i.e. attenuated, the ABTS radical at 50 μg/ml and 250 μg/ml. ABTS radical is also attenuated, in a dose-dependent manner, by 50 μg/ml and 250 μg/ml of soy isoflavones (83% and 91% inactivation respectively). Furthermore, taurine and L-carnitine did not display any effect on ABTS radical inhibition. ABTS radical is also almost completely inhibited by 50 μg/ml and 250 μg/ml of the SPLT mixture (containing respectively 12.5 μg/ml and 62.5 μg/ml of each ingredient). This inhibition percentage is equivalent to that obtained with 50 μg/ml of pomegranate extract alone.

d. DPPH Antioxidant Test:

The antiradical activity test is based on the ability of antioxidant molecules to reduce the stable purple radical DPPH (for 1,1 Diphenyl 2 Pycril Hydrazil).

The test was conducted in 96-well plates where 190 μl of DPPH solution (150 μM) in ethanol were mixed with 10 μl of sample solution prepared in a PBS buffer at the chosen concentrations.

The absorbance of the mixture is read at 540 nm every 5 minutes for 30 minutes at ambient temperature. After reduction, the color of the solution disappears (change from a violet color to a light yellow color).

The reduction (referred to herein as “inhibition” or “quenching”) percentage reflects the free radical scavenging capacity of the test sample.

Trolox is also used as a positive control.

The results are given as a DPPH radical inhibition percentage. It should be noted that, with pomegranate extract at a 250 μg/ml concentration, a precipitate was observed after incubation with DPPH, preventing a relevant absorbance reading.

The DPPH test results are reproduced in table B hereinafter:

TABLE B

DPPH TEST

| | | | Standard |
|---|---|---|---|
| Sample | | % DPPH | deviation |
| Control | | 0.00 | 3.74 |
| Soy isoflavones | 1 | 1.65 | 4.93 |
| (μg/ml) | 50 | 2.35 | 4.62 |
| | 250 | 10.98 | 4.56 |
| Pomegranate (μg/ml) | 1 | 11.53 | 7.02 |
| | 50 | 55.26 | 3.39 |
| | 250 | NC | NC |
| L-carnitine (μg/ml) | 1 | 6.74 | 5.45 |
| | 50 | 4.97 | 5.8 |
| | 250 | 14.94 | 5.09 |
| Taurine (μg/ml) | 1 | 4.95 | 6.03 |
| | 50 | 10.84 | 5.12 |
| | 250 | 25.09 | 4.8 |
| SPCT mixture (μg/ml) | 1 | 5.26 | 5.29 |
| | 50 | 44.49 | 1.88 |
| | 250 | 50.98 | 5.01 |
| Trolox (μM) | 5 | 4.21 | 3.62 |
| | 10 | 9.91 | 2.75 |
| | 15 | 17.00 | 2.83 |
| | 20 | 23.49 | 3.79 |
| | 25 | 28.6 | 2.75 |
| | 30 | 33.06 | 2.37 |
| | 40 | 41.18 | 1.48 |

As demonstrated in table B above, both the pomegranate extract and the SPCT mixture at 50 μg/ml display a significant scavenging effect on DPPH free radicals (approximately 50%), similar to the activity obtained with the 40 μM Trolox control.

Soy isoflavone extract, L-carnitine and taurine exhibit an inhibitory effect on DPPH radical quenching of 11%, 15% and 24% respectively at 250 μg/ml.

Conclusion:

It was demonstrated that H₂O₂ is capable of generating oxidative damage resulting in CnAOEC cell death for concentrations greater than 2 mM. At concentrations less than 2 mM, a slight increase in metabolic activity was observed. It has already been reported that, in some cases, cells surviving oxidative stress induced by H₂O₂ developed hypertrophy associated with the activation of various metabolic pathways.

Following the development of the test on cells, the antioxidant potential of the four substances (i.e. L-carnitine, taurine, extracts of pomegranate and soy isoflavones) was studied in canine endothelial cells.

Pomegranate extract alone or in combination demonstrated a cytoprotective effect. This effect of pomegranate observed on “oxidized” CnAOEC cells is in line with the previous results determined with human endothelial cells and macrophages subjected to H₂O₂, demonstrating that pomegranate extract has a cytoprotective effect in addition to the antioxidant activity. The soy isoflavone extract, L-carnitine and taurine used alone did not display a protective effect on CnAOEC cells under the influence of oxidative stress in this model. The absence of a cytoprotective effect of L-carnitine and taurine suggests that the protective activity of these compounds in the cardiovascular sphere could be associated with different mechanisms of action.

The combination of natural substances (each at equivalent dose) displayed a significant protective effect on CnAOEC cells subjected to oxidative stress, increasing cell viability. This effect appears to be essentially due to the antioxidant properties of pomegranate and/or a potential synergistic effect of the substances. It is important to note that the presence of further substances in the mixture does not appear to antagonize the activity of pomegranate.

Considering that taurine and carnitine is routinely used in dogs suffering from heart disease, it is important to establish that there is no inhibitory effect on other antioxidant actions when they are combined. Indeed, as demonstrated in this study, it would appear that the combination of the various substances induces an improvement in the effect, such as for example in the 50 μg/ml SPCT mixture (Mix), i.e. the mixture of the 4 substances. In this mixture of 50 μg/ml of SPCT, pomegranate is merely at 12.5 μg/ml and this mixture induces a 41% increase in viability comparable to the value (i.e. 35%) of pomegranate alone at 50 μg/ml.

In the free radical scavenging test, pomegranate extract was the only substance displaying a high scavenging activity both on ABTS and DPPH radicals. These results are in line with the results of prior studies on pomegranate extract. The preparations (extracts or juices) using different parts (arils, barks or whole fruits) extracted from pomegranate displayed various antioxidant potentials, evaluated with a plurality of radical scavenging methods.

The antioxidant activity of pomegranate extract would appear to be associated with a high punicoside content (≧40%). Soy isoflavones only displayed a direct radical scavenging activity with the TEAC test and a weak effect on the DPPH tests.

Finally, L-carnitine and taurine do not display any scavenging activity with ABTS with a low (carnitine) to moderate (taurine) activity with the DPPH test.

In sum, the results obtained from the acellular and cellular systems demonstrate that taurine, L-carnitine, extracts of pomegranate and soy isoflavones have antioxidant and cytoprotective activities via different mechanisms of action.

A multi-component treatment appears to be the best approach for reducing the onset and progression of canine heart diseases associated with oxidative stress such as heart failure, after chronic mitral insufficiency (CMVI), and various forms of cardiomyopathy.

### Example 3

### Example of an Oral Composition According to the Invention

| | Content in % |
|---|---|
| | (mass/total mass) |
| Active ingredients | |
| L-carnitine-L-tartrate | 21.8 |
| Taurine | 14.7 |
| Pomegranate extract | 0.9 |
| Concentrated soy isoflavone | 1.5 |
| extracts | |
| Excipients | |
| Binding agent | 32.2 |
| Granulation agent | 2.8 |
| Lubricant | 6.0 |
| Feeding agent | 20.1 |

Conclusion:

The pomegranate extract and the mixture of the four substances displayed a strong antiradical, antioxidant, anti-proliferation and anti-apoptotic action in CnAOEC cells.

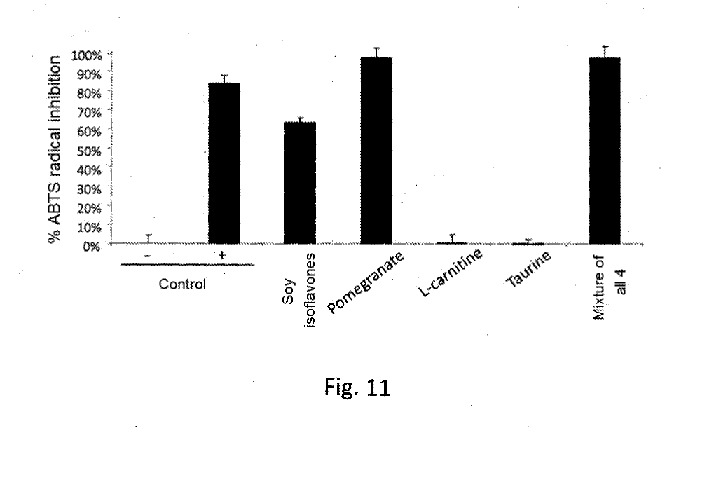

The antiradical actions in terms of pomegranate extracts and the mixture of the four substances being comparable, as demonstrated in FIG. 11, it would thus appear that carnitine, taurine and soy isoflavones have no antagonistic effect on the action of pomegranate extract.

As demonstrated by the results of the present examples, the fact that the antioxidant and anti-proliferation activities of the mixture of the four substances on CnAOEC cells are markedly stronger than those of pomegranate extract alone or of each of the other test substances taken separately indicates that there is a synergistic effect associated with this mixture.

1. A product for oral administration intended for a pet, comprising:
extracts of punica granatum (pomegranate);
soy isoflavone extracts;
L-carnitine; and
taurine.

2. (canceled)

3. The product according to claim 1, comprising:
between 5% and 15% by weight of the total weight of the product of L-carnitine;
between 10% and 20% by weight of the total weight of the product of taurine;
between 0.1% and 5% by weight of the total weight of the product of extracts of punica granatum (pomegranate); and
between 0.5% and 5% by weight of the total weight of the product of concentrated soy isoflavone extracts.

4-5. (canceled)

6. A method of treating or protecting cardiovascular disease, comprising administering the product according to claim 1 for treating or protecting cardiovascular disease.

7. (canceled)

8. A method of supporting cardiac function in the case of chronic heart failure, comprising administering the product according to claim 1, for supporting cardiac function in the case of chronic heart failure.

9. A method of treating or protecting cardiovascular disease, comprising administering a product for oral administration intended for a pet, said product comprising extracts of punica granatum (pomegranate) and further comprising at least two additional antioxidant compounds chosen from:
soy isoflavone extracts;
L-carnitine; and
taurine,
wherein the pet is a canid or felid.

10. (canceled)

11. The method according to claim 9, wherein the animal is a canid selected from dogs of the following breeds: Poodle, Chihuahua, Bichon, Yorkshire, King Charles Spaniel, Pekinese, Pinscher, Keeshond, Spaniels, English Springer Spaniel, Pomeranian, Basset, Beagle, Westie, Whippet, Terriers, Fox Terrier, Yorkshire Terrier.

12. The method according to claim 9, wherein the product is administered daily for a period of at least 1, 2, 3, 4, 5 or 6 months.

13. The method according to claim 9, wherein the product is administered at a dose between 0.0001 mg/kg and 350 mg/kg.

14. The method according to claim 9, wherein the product is in the form of a tablet, capsule, sugar-coated tablet, powder, granule, syrup, suspension, solution or emulsion.

15. A method comprising administering the product according to claim 1 as a complete food.

16. The method according to claim 15, for helping to control cardiovascular disease.

17. The method according to claim 15, wherein the pet is a canid or a felid.

18. A method comprising administering the product according to claim 1 as a supplementary or health food.

19. The method according to claim 18, for helping to control cardiovascular disease.

20. The method according to claim 18, wherein the pet is a canid or a felid.
